(19) United States
(12) Patent Application Publication
(10) Pub. No.: US 2026/0144752 A1
(43) Pub. Date: May 28, 2026

(54) Multilamellar Vesicles and Methods and Uses Thereof

(71) Applicants: ChromoLogic LLC, Monrovia, CA (US); University of Pittsburgh - Of the Commonwealth System of Higher Education, Pittsburgh, PA (US)

(72) Inventors: Thomas A. Miller, Monrovia, CA (US); Ricky Whitener, Monrovia, CA (US); Claude J. Rogers, Monrovia, CA (US); Naresh Menon, Monrovia, CA (US); Joel S. Greenberger, Pittsburgh, PA (US); Michael Epperly, Pittsburgh, PA (US); Peter Wipf, Pittsburgh, PA (US)

(73) Assignees: ChromoLogic LLC, Monrovia, CA (US); University of Pittsburgh - Of the Commonwealth System of Higher Education, Pittsburgh, PA (US)

(21) Appl. No.: 19/121,109

(22) PCT Filed: Oct. 16, 2023
(86) PCT No.: PCT/US23/77006

§ 371 Date: Apr. 14, 2025

Related U.S. Application Data
(60) Provisional application No. 63/379,629, filed on Oct. 14, 2022.

(51) Int. Cl. A61K 9/1271; A61K 31/445

(52) U.S. Cl. CPC A61K 9/1271; A61K 31/445

(57) ABSTRACT

Multilamellar vesicles (MLVs), methods of their manufacture, and applications thereof to the administration of compounds are provided. MLVs packaged with one or more medicinal compounds, including drugs are also provided. Methods for modifying MLVs to allow for targeting and/or selective accumulation of MLVs to certain tissues are also provided.

### CROSS-REFERENCE TO RELATED CASES

This application claims priority to U.S. Provisional Application No. 63/379,629, filed Oct. 14, 2022, this disclosure of which is incorporated in its entirety by reference.

### STATEMENT REGARDING FEDERALLY SPONSORED RESEARCH OR DEVELOPMENT

This invention was made with government support under Contract Nos. W81XWH-18-C-0358, W81XWH-17-C-0221, W81XWH-21-P0091 & W81XWH22C0096 awarded by the Defense Health Agency and 80NSSC21C0146 awarded by the National Aeronautics and Space Administration. The government has certain rights in this invention.

### FIELD OF THE INVENTION

The present invention is directed to extracellular vesicles, including methods of synthesis and applications thereof; more particularly, multilamellar vesicles configured to carry compounds with low aqueous solubility, including medicaments delivered via an multilamellar vesicle as well as method to deliver an multilamellar vesicle to a patient.

### BACKGROUND OF THE INVENTION

Exposure to ionizing radiation (IR) can result in death or significant morbidity depending on the dose and the amount of tissue in the radiation field. Total body irradiation (TBI) in doses≥2 Gy can result in acute radiation syndrome (ARS) and result in morbidity within weeks. Shielding significantly reduces the risk of ARS lethality, but delayed effects of radiation exposure, characterized by chronic organ dysfunction and persistent inflammation, can still persist. Even therapeutic exposure to IR can result in tissue toxicity that can manifest into acute or delayed injuries in patients.

At harmful doses, IR results in DNA damage, membrane damage, mitochondrial dysfunction, and the accumulation of reactive oxygen species (ROS). Radiation-induced mitochondrial dysfunction is associated with metabolic oxidative stress that leads to lethal cellular injury and cell death. ROS induce lipid peroxidation in the mitochondrial membrane begins a chain of reactions of oxidative degradation of lipids, where ROS free radicals “steal” electrons from the lipids in cell membranes, resulting in cell damage. The cascade leads to increased mitochondrial membrane permeability, release of cytochrome c into the cytoplasm, and activation of caspase 3, resulting in apoptotic cell death. These effects can lead to inflammation, genomic instability, and tissue injury.

Therapeutics that can prevent or mitigate the harmful effects of IR are therefore needed. When taken before or after IR exposure, therapeutics can mitigate ROS, and therefore can be an effective stage for mitigating radiation-induced injury. However, there are challenges in the administration of the therapeutics; such as maintaining stability or solubility when delivering therapeutics into lipid or aqueous environments.

### SUMMARY OF THE INVENTION

This summary is meant to provide some examples and is not intended to be limiting of the scope of the invention in any way. Any feature included in an example of this summary is not required by the claims, unless the claims explicitly recite the feature. Features and steps described elsewhere in this disclosure may be included in the examples summarized here. The features and steps described here and elsewhere can be combined in a variety of ways.

In some aspects, the techniques described herein relate to a composition for the delivery of a compound, including at least one multilamellar vesicle (MLV), where the at least one MLV is loaded with a compound.

In some aspects, the techniques described herein relate to a composition, where the compound is hydrophobic or lipophilic.

In some aspects, the techniques described herein relate to a composition, where the compound is ameliorative of at least one of traumatic brain injury, reactive oxygen species damage, DNA double-strand breaks, and ionizing radiation damage.

In some aspects, the techniques described herein relate to a composition, where the compound is JP4-039.

In some aspects, the techniques described herein relate to a composition, where the MLV is included of multiple layers of lipid bilayers.

In some aspects, the techniques described herein relate to a composition, where the multiple layers are cross-linked.

In some aspects, the techniques described herein relate to a composition, where the average size of the MLVs is between approximately 100 nm to approximately 500 nm.

In some aspects, the techniques described herein relate to a composition, where the average size of the MLVs is approximately 250 nm.

In some aspects, the techniques described herein relate to a composition, where the MLVs possess a polydispersity index of less than 0.7.

In some aspects, the techniques described herein relate to a composition, where the MLVs possess a polydispersity index of approximately 0.2.

In some aspects, the techniques described herein relate to a method of manufacturing multilamellar vesicles (MLVs), including forming MLVs from adjacent lipid bilayers, and loading the MLVs with a payload compound.

In some aspects, the techniques described herein relate to a method, where forming MLVs includes cross-linking the adjacent lipid bilayers, dehydrating and rehydrating the adjacent lipid bilayers to form the MLVs.

In some aspects, the techniques described herein relate to a method, further including sonicating the MLVs to alter once characteristic selected from a size of the MLVs, a size distribution of the MLVs, and combinations thereof.

In some aspects, the techniques described herein relate to a method, where the average size of the MLVs is between approximately 100 nm to approximately 500 nm.

In some aspects, the techniques described herein relate to a method, where the average size of the MLVs is approximately 250 nm.

In some aspects, the techniques described herein relate to a method, where the MLVs possess a polydispersity index of less than 0.7.

In some aspects, the techniques described herein relate to a method, where the MLVs possess a polydispersity index of approximately 0.2.

In some aspects, the techniques described herein relate to a method, where the payload compound is hydrophobic or lipophilic.

In some aspects, the techniques described herein relate to a method, where the compound is ameliorative of at least one of traumatic brain injury, reactive oxygen species damage, DNA double-strand breaks, and ionizing radiation damage.

In some aspects, the techniques described herein relate to a method, where the compound is JP4-039.

In some aspects, the techniques described herein relate to a method, further including surface modifying the MLVs.

In some aspects, the techniques described herein relate to a method, where the surface modification is selected from a lipid, a carbohydrate, a peptide, and combinations thereof.

In some aspects, the techniques described herein relate to a method, where the surface modification is PEGylation.

In some aspects, the techniques described herein relate to a method, further including packaging the MLVs.

In some aspects, the techniques described herein relate to a method, where packaging includes lyophilizing the MLVs, and adding the MLVs to a vial.

In some aspects, the techniques described herein relate to a method, where packaging includes adding the MLVs to a syringe or autoinjector.

In some aspects, the techniques described herein relate to a medical article containing MLVs including a vessel containing a formulation of MLVs.

In some aspects, the techniques described herein relate to a medical article, where the vessel is selected from a vial, a bottle, a syringe, and an autoinjector.

In some aspects, the techniques described herein relate to a medical article, where the formulation is a lyophilized powder.

In some aspects, the techniques described herein relate to a medical article, where the formulation is a liquid suspension.

In some aspects, the techniques described herein relate to a medical article, where the MLVs in the formulation contain a compound.

In some aspects, the techniques described herein relate to a medical article, where the compound is hydrophobic or lipophilic.

In some aspects, the techniques described herein relate to a medical article, where the compound is ameliorative of at least one of traumatic brain injury, reactive oxygen species damage, DNA double-strand breaks, and ionizing radiation damage.

In some aspects, the techniques described herein relate to a medical article, where the compound is JP4-039.

In some aspects, the techniques described herein relate to a method of treating an individual, including obtaining a medical formulation of MLVs, and administering a dose of the formulation to an individual.

In some aspects, the techniques described herein relate to a method, where the formulation is a lyophilized powder.

In some aspects, the techniques described herein relate to a method, where the formulation is a liquid suspension.

In some aspects, the techniques described herein relate to a method, where the MLVs in the formulation contain a compound.

In some aspects, the techniques described herein relate to a method, where the compound is hydrophobic or lipophilic.

In some aspects, the techniques described herein relate to a method, where the compound is ameliorative of at least one of traumatic brain injury, reactive oxygen species damage, DNA double-strand breaks, and ionizing radiation damage.

In some aspects, the techniques described herein relate to a method, where the compound is JP4-039.

In some aspects, the techniques described herein relate to a method, where the formulation is packaged in a vessel.

In some aspects, the techniques described herein relate to a method, where the vessel is selected from a vial, a bottle, a syringe, and an autoinjector.

In some aspects, the techniques described herein relate to a method, further including preparing a dose of the formulation.

In some aspects, the techniques described herein relate to a method, where preparing includes reconstituting a lyophilized powder of the formulation.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

These and other features and advantages of the present invention will be better understood by reference to the following detailed description when considered in conjunction with the accompanying drawings where:

FIG. 1A provides an illustration of the structure of the compound JP4-039 in accordance with many embodiments.

FIG. 1B illustrates a diagram of an MLV containing a small compound, JP4-039 (JP4-039-MLV) in accordance with various embodiments.

FIG. 1C illustrates an exemplary transmission electron micrograph of an MLV in accordance with various embodiments.

FIG. 2 provides an exemplary method for preparing MLVs in accordance with various embodiments.

FIGS. 3A-3D illustrate exemplary data characterizing MLVs in accordance with various embodiments.

FIGS. 4A-4B illustrate exemplary data from an embodiment showing the ability to reduce DNA Double Strand Breaks in accordance with various embodiments.

FIGS. 5A-5C provide exemplary data of an embodiment showing reduction of lonizing Radiation-induced cellular dysregulation in accordance with various embodiments.

FIGS. 6A-6B provide exemplary data showing increased survivability of cells exposed to lonizing Radiation in accordance with various embodiments.

FIG. 7 illustrates exemplary cell viability data of MLVs in accordance with various embodiments.

FIG. 8 illustrates exemplary fluorescence micrographs showing MLV accumulation on cell surfaces in accordance with various embodiments.

FIG. 9 provides an exemplary method for administering MLVs to an individual in accordance with various embodiments.

### DETAILED DISCLOSURE OF THE INVENTION

Turning now to the diagrams and figures, embodiments of the invention are generally directed to multilamellar vesicles (MLVs), methods of their manufacture, and applications thereof. Many embodiments are directed to MLVs packaged with one or more medicinal compounds, including drugs. Further embodiments include modifications to MLVs to allow for targeting and/or selective accumulation of MLVs to certain tissues. MLV structures, in accordance with many embodiments, may be used as a delivery platform for hydrophobic and/or lipophilic compounds, including JP4-039. Various embodiments describe the synthesis and in vitro and in vivo evaluation of MLV encapsulated JP4-0439 (JP4-093-MLV). Further embodiments are directed to systems and methods to administer JP4-039-MLV, including as a radiation countermeasure and easily deployable inside or outside (e.g., for prophylactic and/or field use) of medical facilities.

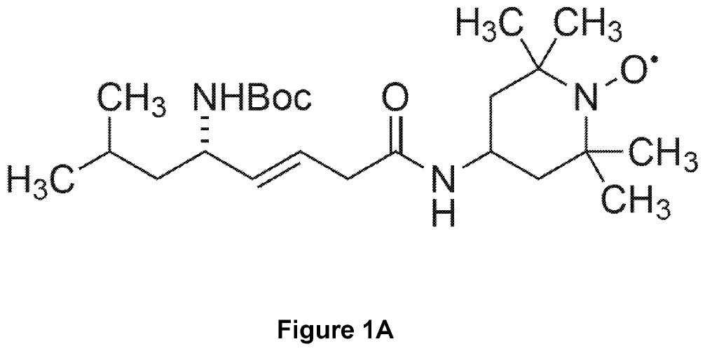

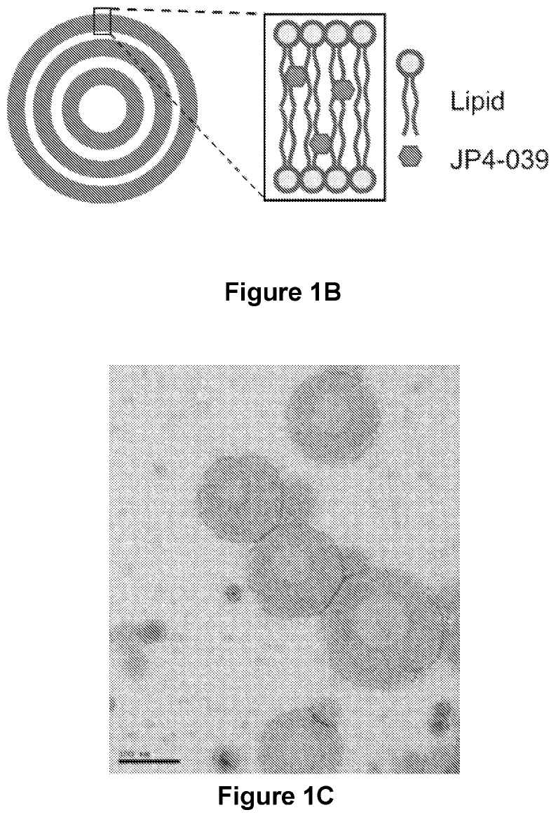

An exemplary radiation countermeasure designed to deliver a nitroxide functional group to a mitochondria to scavenge radicals generated by IR is the small compound, JP4-039 (FIG. 1A). (See e.g., Rwigema, J. C. M., et al. Two strategies for the development of mitochondrion-targeted small molecule radiation damage mitigators. Int. J. Radiat. Oncol. Biol. Phys. 2011, 80, 860-868; Hoye, A. T. et al., Targeting mitochondria. Acc. Chem. Res. 2008, 41, 87-97; and Fink, M. P.; et al., Hemigramicidin-TEMPO conjugates: novel mitochondria-targeted anti-oxidants. Biochem. Pharmacol. 2007, 74, 801-809; the disclosures of which are hereby incorporated by reference in their entireties.) The functional group in JP4-039 is able to cycle between nitroxide, hydroxylamine, and nitroxonium redox states. (See e.g., Tyurina, Y. Y.; et al., Oxidative lipidomics of hyperoxic acute lung injury: mass spectrometric characterization of cardiolipin and phosphatidylserine peroxidation. Am. J. Phys. Lung Cell. Mol. Physiol. 2010, 299, L73-85; the disclosure of which is hereby incorporated by reference in its entirety.) JP4-039 has been shown to be an effective radiation countermeasure in a variety of contexts, including increasing survival following hematopoietic-or gastrointestinal-ARS-inducing doses of TBI, the prevention of radiation injury when administered before exposure, and reducing toxicity to normal tissues in the radiation field during cancer therapy. (See e.g., Epperly, M. W.; et al., Effectiveness of analogs of the GS-nitroxide, JP4-039, as total body irradiation mitigators. In Vivo 2017, 31, 39-43; Goff, J. P.; et al., Radiobiologic effects of GS-nitroxide (JP4-039) on the hematopoietic syndrome. In Vivo 2011, 25, 315-323; Wei, L.; et al., The GS-nitroxide JP4-039 improves intestinal barrier and stem cell recovery in irradiated mice. Sci. Rep. 2018, 8, 2072; Bernard, M. E.; et al., GS-nitroxide (JP4-039)-mediated radioprotection of human Fanconi anemia cell lines. Radiat. Res. 2011, 176, 603-612; Epperly, M. W.; et al., Intraesophageal administration of GS-nitroxide (JP4-039) protects against ionizing irradiation-induced esophagitis. In Vivo 2010, 24, 811-819; Berhane, H.; et al., Amelioration of radiation-induced oral cavity mucositis and distant bone marrow suppression in Fanconi anemia Fancd2−/− (FVB/N) mice by intraoral GS-nitroxide JP4-039. Radiat. Res. 2014, 182, 35-49; Shinde, A.; et al., Intraoral mitochondrial-targeted GS-nitroxide, JP4-039, radioprotects normal tissue in tumor-bearing radiosensitive Fancd2(−/−) (C57BL/6) mice. Radiat. Res. 2016, 185, 134-150; and Quinn, T. J.; et al., Amelioration of mucositis in proton therapy of Fanconi anemia Fanca(−/−) mice by JP4-039. In Vivo 2019, 33, 1757-1766; the disclosures of which are hereby incorporated by reference in their entireties.)

In vivo radiation survival studies with JP4-039 suggest that the drug is effective 24 h prior to 72 h after irradiation, and that the long-term release nature of MLV drug delivery may be beneficial. (See e.g., Greenberger, J. S.; et al.; Strategies for discovery of small molecule radiation protectors and radiation mitigators. Front. Oncol. 2011, 1, 59; the disclosure of which is hereby incorporated by reference in its entirety.) However, due to hydrophobicity of JP4-039, it has very low solubility in an aqueous environment making the administration of JP4-039 outside of a laboratory setting impractical using conventional techniques.

Embodiments of the disclosure are directed to MLVs and methods of forming MLVs as vehicles for the administration of JP4-039 and other hydrophobic and/or lipophilic compounds, as well as to methods of treatment involving the administration of such MLV encapsulated small compounds for the treatment of various disorders. Further embodiments may include surface modifications (e.g., PEGylation) and/or internal modifications (e.g., cross-linking), which can improve delivery of payloads (e.g., JP4-039 or other compounds) to tissues (including the brain following traumatic injury), improving stability/half-life of the payload. (See e.g., Whitener, R.; et al.; Localization of multilamellar vesicle nanoparticles to injured brain tissue in a controlled cortical impact injury model of traumatic brain injury in rodents. Neurotrauma Rep. 2022, 3, 158-167; the disclosure of which is hereby incorporated by reference in its entirety.)

### Preparation of MLV NPs

Multilamellar Vesicles (MLVs) as illustrated in FIG. 1C are a type of vesicle that consist of multiple lamellar phases. A vesicle refers to a liquid or cytoplasm enclosed in a lipid bilayer. A lamellar phase refers to essentially flat and infinite bilayers of amphiphilic molecules alternated by a bulk polar liquid, such as water. MLVs are a subtype of liposomes, which are small artificial vesicles, spherical in shape, having at least one lipid bilayer. Liposomes can vary in size and may contain small amounts of other molecules as illustrates in FIG. 1B. In the case of MLVs, the vesicles have an onion-like structure. Multiple lamellar vesicles form one inside the other with decreasing size, creating a multilamellar structure of concentric lipid spheres (FIG. 1B). These vesicles, which can be of variable sizes up to several micrometers, are composed of many concentric amphiphilic lipid bilayers, similar to the layers of an onion. The lipid layers of these vesicles detach during agitation and self-close to form MLVs, which prevents interaction with the bilayer core.

The characteristic of MLVs, including MLV nanoparticles (NPs), to reform allows them to encapsulate hydrophobic small compounds like JP4-039 within the lipid bilayers as illustrated by FIG. 1B. This encapsulation process can be confirmed via microscopy techniques, such as transmission electron microscopy as illustrated in FIG. 1C, and reveals the multiple lipid bilayers of the MLVs.

Alternative drug delivery vehicles face challenges in the administration of their therapeutics; such as maintaining stability or solubility when delivering therapeutics into lipid or aqueous environments. Once encapsulated, within an MLV payload compounds can be released in vivo into hydrophobic elements, such as cell membranes, in tissues and circulation. This is particularly useful for the administration of hydrophobic or lipophilic pharmaceutical drugs and nutrients, and makes MLVs an effective drug delivery vehicle for such therapeutics.

In accordance with one embodiment, an exemplary therapeutic GS-nitroxide JP4-039 as illustrated in FIG. 1A, is a mitochondrially targeted nitroxide that assists mitochondria in combating irradiation-induced cell death by reducing oxidative stress. However, due to the hydrophobicity of JP4-039, it has very low solubility in an aqueous environment making the administration of JP4-039 outside of a laboratory setting impractical using conventional techniques. However, encapsulating JP4-039 in an MLV (JP4-039-MLV) allow for its use as a practical therapeutic. MLV encapsulation reduces the need for frequent administration, allows for longer term storage, and can reduce negative side effects.

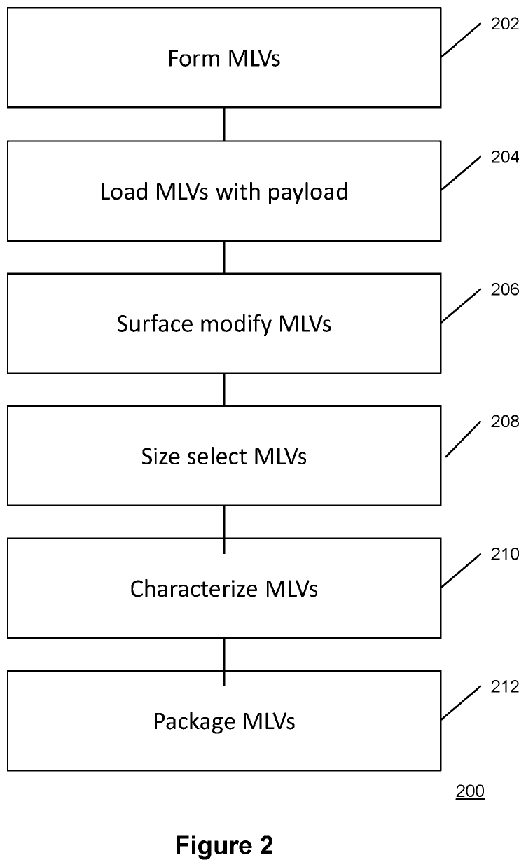

MLVs, in accordance with various embodiments can be prepared in a variety of ways. In many embodiments MLVs are generated using a liposomal formulation technique with improved drug bioavailability and particle stability. FIG. 2 provides an exemplary method 200 for preparing various embodiments. In many embodiments, MLVs are formed at 202. In many embodiments, MLVs are formed through covalently crosslinking functionalized head groups of adjacent lipid bilayers. Numerous embodiments use a multistep procedure based on conventional dehydration-rehydration methods by incorporation of a thiol-reactive maleimide head-group lipid, covalently binding through the use of dithiothreitol. (See e.g., Moon, J. J.; et al., Interbilayer-crosslinked multilamellar vesicles as synthetic vaccines for potent humoral and cellular immune responses. Nat. Mater. 2011, 10, 243-251 and 23. Joo, K. I.; et al.; Crosslinked multilamellar liposomes for controlled delivery of anticancer drugs. Biomaterials 2013, 34, 3098-3109; the disclosures of which are hereby incorporated by reference in their entireties.)

The lipid composition of bilayers can further be altered for various properties, including stability, ability to contain a specific compound, and/or any other reason. Lipids used in bilayers can be saturated, unsaturated, and/or modified to alter any such property of the lipid bilayers and/or resulting MLVs.

Adjusting parameters of the formation methods can alter size distribution, average size, and/or other parameters. Some embodiments can further use sonication to adjust size and/or size distribution of MLVs. Various embodiments possess an average size of between approximately 100 nm to approximately 500 nm, and in some embodiments between approximately 200 nm to approximately 300 nm, including average sizes of approximately 125 nm, approximately 150 nm, approximately 175 nm, approximately 200 nm, approximately 225 nm, approximately 250 nm, approximately 275 nm, approximately 300 nm, approximately 350 nm, approximately 400 nm, approximately 450 nm, and approximately 500 nm. Additional embodiment can also have a polydispersity index (PDI) of less than 0.7, including a PDI approximately 0.5, 0.4, 0.35, 0.3, 0.25, 0.2, 0.18, 0.16, 0.15, 0.12, 0.1, 0.05.

Further embodiments alter the amount of inter-membrane crosslinking present in the MLVs. Crosslinking lipid bilayers may help stabilize MLVs. MLV stability may alter dissolution or breakdown constants of MLVs and/or release of a compound contained within the MLVs. Thus, increasing the level of crosslinking may increase stability and/or slow release of a contained compound, while decreasing the level of crosslinking may decrease stability and/or increase compound release. As such, various embodiments are customizable or tunable for a specific release profile.

At 204, further embodiments load MLVs with a payload compound. Such loading can include known techniques to insert the payload, including using certain solvents, surfactants to increase MLV permeability to allow payload compounds to enter the MLVs. Further embodiments use physical means to assist in loading, such as increased temperature and/or electroporation. In certain embodiments, carrier molecules that can increase aqueous solubility of payload compounds. Certain carrier molecules may allow for increased loading of payload compounds into MLVs. Certain embodiments may combine MLV formation 202 with MLV loading 204, such that payload compounds are included with lipid bilayers, allowing for simultaneous forming 202 and loading 204.

Further embodiments add surface modifications at 206. Surface modification can proceed via various known means. Such surface modifications can include carbohydrates, lipids, peptides, and combinations thereof. In many embodiments, surface modification can include any molecule that includes a thiol group and/or a group that is derivable to a thiol. Exemplary modifications include PEGylation, addition of antigens, antibodies, and/or other surface markers. Certain modifications (e.g., PEGylation) can improve clearance properties of MLVs, while other modifications can increase tissue targeting (e.g., increasing tissue specificity), immune system evasion, immune system stimulation, and/or any other relevant property that may be desirable. In certain embodiments, surface modifications are added to lipid bilayers prior to MLV formation 202, such that formation of MLVs at 202 already includes surface modifications.

Some embodiments perform size select for MLVs of certain sizes at 208. Size selection of certain embodiments can include filtering MLVs, such as via high-pass and/or low-pass filters to remove MLVs below or above a certain threshold size. Such filtering can alter average size and/or size distribution. Size selection can occur before and/or after loading 204 and/or surface modification 206, as such alterations may alter size of some embodiments of MLVs.

Additional embodiments characterize MLVs at 210. Various embodiments use dynamic light scattering to measure size distribution of MLVs. Additional embodiments use additional techniques, such as enzyme-linked immunosorbent assay (ELISA), fluorescence, flow cytometry, and/or other methods to characterize sizes of MLVs. Additional methods exist to identify loading concentration of the payload compound(s), such as high-performance liquid chromatography (HPLC).

At 212, many embodiments package MLVs. Packaging in some embodiments includes processing the MLVs, such as by dehydration, lyophilization, etc., while certain embodiments mix MLVs in a buffer, such as a buffer for delivery as a medicament. Some embodiments include additional materials to aid in medical use, such as buffers, flavorants, lubricants, colorants, fillers, etc., which can assist for medical use. Various embodiments package MLVs with or without additional materials as a dry powder that can be reconstituted later with an appropriate diluent (e.g., water, buffer, etc.), while some embodiments package MLVs as a liquid solution that can be administered as-is. Certain embodiments package MLVs into an autoinjector, which can reconstitute a dry MLV prior to injection.

### Characteristics of MLVs

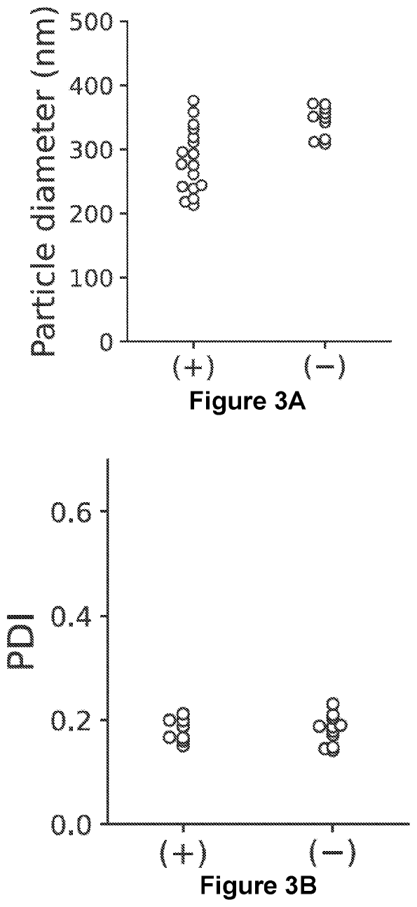

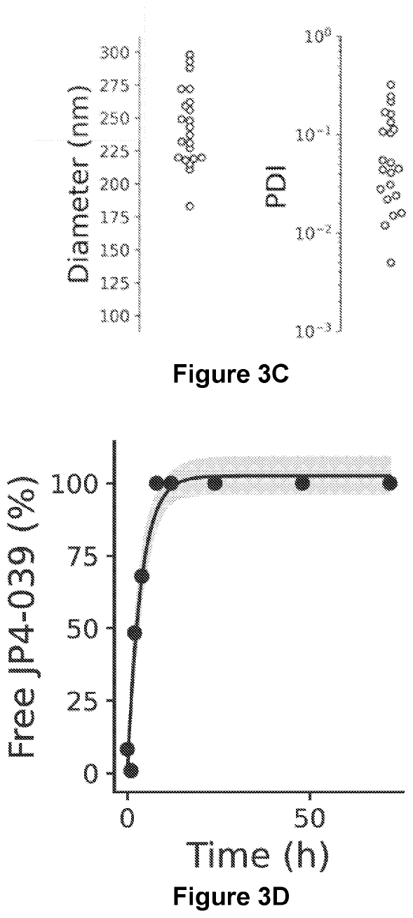

In many embodiments, MLVs can be generated to specific sizes via size selection, modifications to formation protocols allowing for different sizes, and/or additional steps to (e.g., sonication) to alter sizes. FIGS. 3A-3D illustrate exemplary data characterizing MLVs in accordance with various embodiments. Specifically, FIG. 3A illustrates sizes of loaded (+) and unloaded (−) MLVs, while FIG. 3B illustrates a polydispersity index (PDI) of loaded and unloaded MLVs. In FIGS. 3A-3B, the exemplary embodiments are loaded with JP4-039, and both loaded and unloaded MLVs have similar size distributions (283±50 nm) and PDIs (0.18+0.02). Additionally, FIG. 3C illustrates additional exemplary showing size distribution and PDI, with an average size of approximately 250 nm and very low PDIs (˜0.1). Additionally, FIG. 3D provides an in vitro release rate of JP4-039 in the presence of 1 mM 2-hydroxy-propyl-b-cyclodextrin (CD), indicating complete release of JP4-039 within 12 hours—for comparison, the in vivo half-life of JP4-039 is approximately 6 hours, indicating that many embodiments are capable of sustained or extended release of JP4-039 in MLV formulations. While data is not shown, the JP4-039 was loaded at approximately 85 μg/mg (JP4-039/MLV).

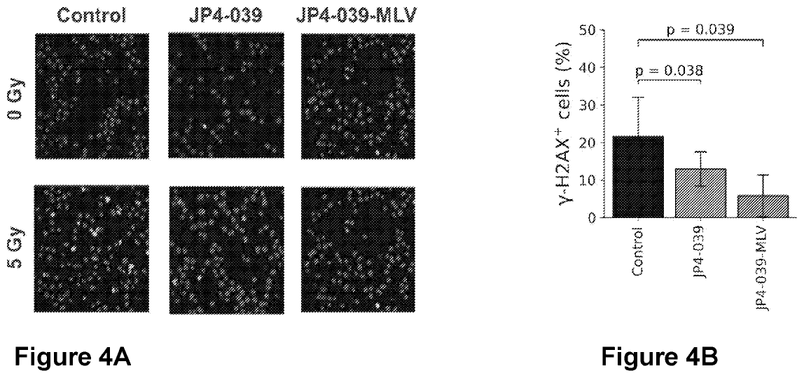

In various embodiments JP4-039-MLVs have the ability to reduce IR-induced DNA double strand breaks (DSBs). IR induces DNA damage resulting in double strand breaks that can result in mitotic failure and cell death. Secondary oxidative stress induces further DNA damage. In response to double strand breaks, the histone H2AX is rapidly phosphorylated, producing γ-H2AX, a standard biomarker of DNA damage. JP4-039 has been shown to reduce the number of γ-H2AX+ intestinal crypt cells in mice following exposure to 9.25 Gy TBI. FIGS. 4A-4B illustrate exemplary data from an embodiment showing the ability to reduce DNA DSBs. Specifically, FIG. 4A provides confocal images of cells treated with DMSO (control), JP4-039, and JP4-039-MLVs prior to irradiation of 0 Gy and 5 Gy. Cell nuclei are stained with DAPI (blue) and γ-H2AX is visualized by immunohistochemistry (green), while FIG. 4B illustrates relative frequency of γ-H2AX+ cells by counting blue puncta and green puncta. FIGS. 4A-4B illustrate that both JP4-039 and JP4-039-MLV nanoparticles protected cells from IR-induced DNA damage.

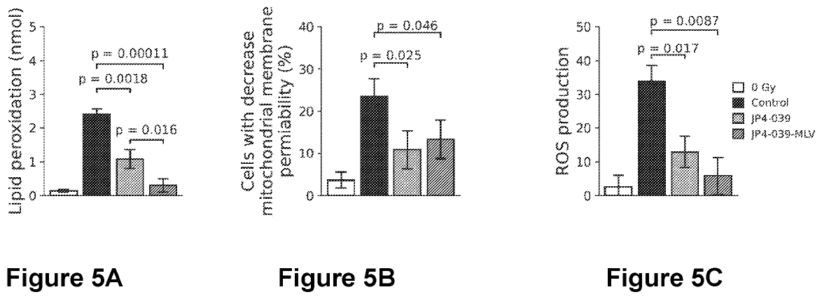

In various embodiments JP4-039-MLV nanoparticles protect cells from IR-induced oxidative stress. In addition to DNA damage, IR-induced oxidative stress can result in lipid peroxidation, mitochondrial membrane permeability, and an increase in ROS concentration. FIGS. 5A-5C provide exemplary data of an embodiment showing reduction of IR-induced cellular dysregulation. Specifically, FIG. 5A illustrates that JP4-039 and JP4-039-MLV nanoparticles reduce levels of lipid peroxidation in cells exposed to 5 Gy IR (FIG. 5A), reduced levels of mitochondrial membrane permeability in cells exposed to 10 Gy IR (FIG. 5B), and reduced ROS levels in cells exposed to 5 Gy IR (FIG. 5C).

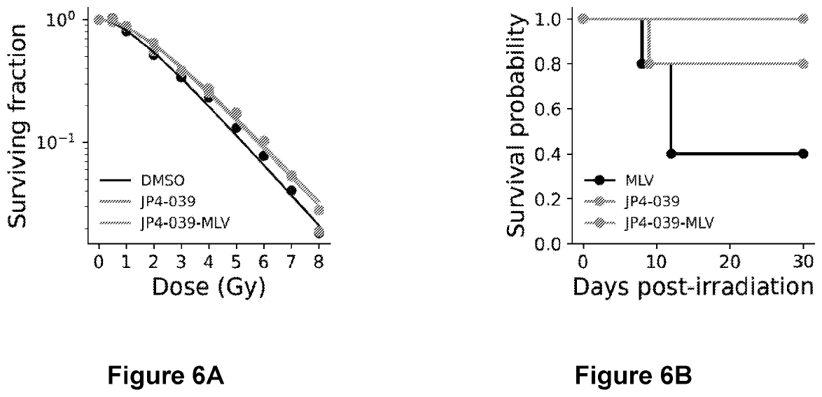

In various embodiments JP4-039-MLV nanoparticles increase cell survival. FIGS. 6A-6B provide exemplary data showing increased survivability of cells exposed to IR. As illustrated in FIG. 6A, JP4-039-MLV increased survivability of cells across a range of IR doses (i.e., 0-8 Gy), while FIG. 6B illustrates increased survivability of mice after exposure of 9.25 Gy irradiation.

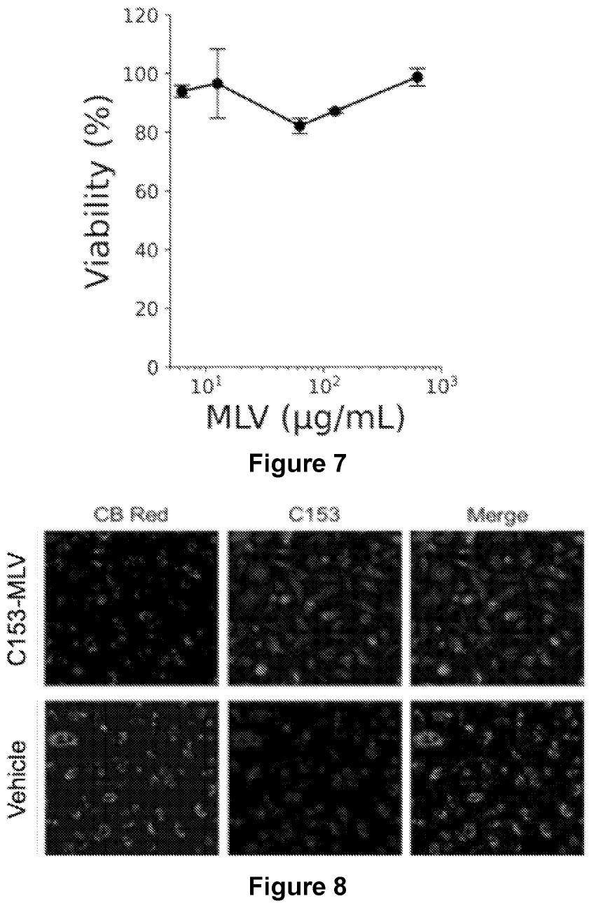

In various embodiments MLVs are not cytotoxic to U-251 MG cells. FIG. 7 illustrates exemplary data showing that MLVs are not cytotoxic. Specifically, FIG. 7 shows cell viability of U-251 MG cells in vitro over three orders of magnitude in MLV nanoparticle concentration. As shown, there is no statistically significant change in cell concentration at a wide range of MLV concentrations: ranging from 10-1000 μg/mL of MLVs.

In various embodiments MLVs localize to the surface of cells in culture. FIG. 8 illustrates exemplary data showing localization to cell surfaces of human brain cerebral microvascular endothelial cells in culture. Specifically, FIG. 8 shows coumarin 153 (C153) and cells in isolation and merged images for C153-loaded MLVs and unloaded MLVs (vehicle). The merged image shows that the fluorescence from C153 colocalizes with the cells, indicating a MLVs localizing to cell surfaces.

### Medical Uses

As noted elsewhere herein, many embodiments are directed to a medical use of MLVs, including JP4-039-MLVs. In some embodiments, the MLVs are formulated as a medicament. Some of these formulations can be MLVs alone or MLVs mixed with one or more additives to assist in delivery, such as flavorants, buffers, lubricants, anti-adherent agents, antioxidants, diluents, fillers, emulsifying agents, glidants, preservatives, adjuvants, and combinations thereof, depending on use or avenue of administration. Administration can include such means as oral, subcutaneous, intravenous, anal, intramuscular, and/or any other method of administration that is effective for purpose.

Certain embodiments formulate MLVs as a dry powder that can be reconstituted at a later date, while other embodiments formulate MLVs in solution. Certain embodiments of formulations are packaged in vials prepared as a dry powder (e.g., to be reconstituted before use), while other embodiments of formulations include suspensions of MLVs. Some formulations package formulations in prepared syringes and/or autoinjectors. Autoinjectors are specialized delivery devices that provide a measured dose of a medicament and can be constructed for single use only or for repeated uses. Certain embodiments of autoinjectors allow for reconstitution of a dose prior to administration, such that through a user action (pushing a button, turning a knob, etc.) a diluent can be mixed with a dry formulation of MLVs. Once reconstituted via mixing, waiting, inverting, etc., the reconstituted MLVs can be injected into a user by the user or by a medical professional.

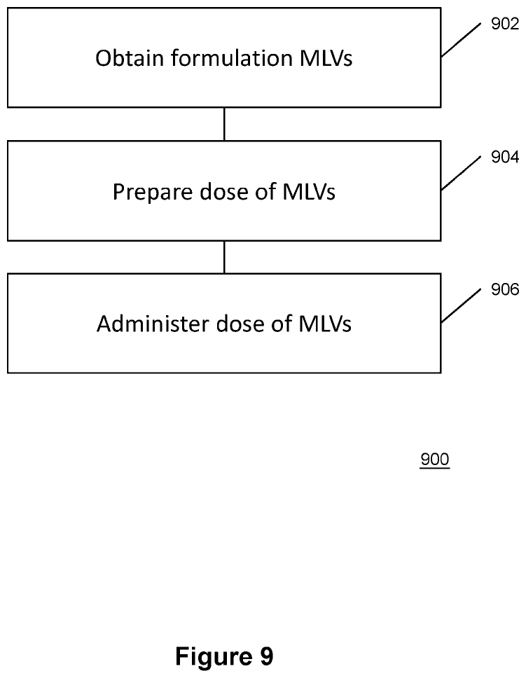

Turning to FIG. 9, many embodiments are directed to methods of medically using MLVs. Specifically, FIG. 9 illustrates a method 900 for administering MLVs to an individual. At 902, many embodiments obtain a medical formulation of MLVs. As noted throughout this disclosure, medical formulations can take many forms and include various payloads, depending on specific use. In certain embodiments, the payload is JP4-039 to use in case of ionizing radiation exposure, traumatic brain injury, and or any other indication where JP4-039 may be an effective treatment. Certain embodiments obtain the formulation as a dry powder, liquid suspension, pill form, etc., as described herein. Such embodiments can be packaged in a vessel or container, such as a vial, bottle, or other container. In some embodiments, the JP4-039-MLV formulation is packaged in an autoinjector or syringe for an injection.

At 904, various embodiments prepare a dose the formulation. Preparation 904 can include reconstituting a dry formulation, thawing a liquid suspension, and/or isolating a specific dose from a larger container (e.g., vial containing multiple doses or removing a set number of pills from a bottle).

Additional embodiments provide a dose to an individual at 906. Depending on specific formulation, the dose can be taken via one of the methods described herein, including oral, subcutaneous, intravenous, anal, intramuscular, and/or any other method of administration. Administration can be taken by a number of users, including medical professionals (e.g., physicians, doctors, nurses, nursing assistants, etc.), a user, and/or other caretaker.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A composition for delivery of a payload compound, comprising at least one multilamellar vesicle (MLV) comprised of multiple layers of lipid bilayers arranged to form a vesicle of more than one lamellar phase, loaded with at least one payload compound that is either hydrophobic or lipophilic, includes JP-039, and is ameliorative of at least one disorder selected from the group of: traumatic brain injury, reactive oxygen species damage, DNA double-strand breaks, and ionizing radiation damage.

2. The composition of claim 1, wherein the multiple layers of lipid bilayers form concentric amphiphilic lipid bilayers.

3. The composition of claim 1, wherein the multiple layers are cross-linked.

4. The composition of claim 1, wherein an average size of the MLVs is between approximately 100 nm to approximately 500 nm.

5. The composition of claim 1, wherein an average size of the MLVs is approximately 250 nm.

6. The composition of claim 1, wherein the MLVs possess a polydispersity index of less than 0.7.

7. The composition of claim 1, wherein the MLVs possess a polydispersity index of approximately 0.2.

8. A method of manufacturing therapeutic MLVs, comprising:
forming a vesicle from adjacent lipid bilayers of more than one lamellar phase, wherein the lamellar phase is comprised of bilayers of amphiphilic molecules; and
loading the vesicle with at least one payload compound, wherein the at least one payload compound is hydrophobic or lipophilic, includes JP4-039, and is ameliorative of at least one of: traumatic brain injury, reactive oxygen species damage, DNA double-strand breaks, and ionizing radiation damage.

9. The method of claim 8, wherein forming the vesicle further comprises:
cross-linking the adjacent lipid bilayers.

10. The method of claim 8, wherein forming the vesicle further comprises dehydrating and rehydrating the adjacent lipid bilayers.

11. The method of claim 8, further comprising sonicating the MLVs to alter one characteristic selected from the group of: a size of the MLVs, a size distribution of the MLVs, and combinations thereof.

12. The method of claim 8, wherein an average size of the MLVs is between approximately 100 nm to approximately 500 nm.

13. The method of claim 8, wherein an average size of the MLVs is approximately 250 nm.

14. The method of claim 8, wherein the MLVs possess a polydispersity index of less than 0.7.

15. The method of claim 8, wherein the MLVs possess a polydispersity index of approximately 0.2.

16. The method of claim 8, further comprising surface modifying the MLVs.

17. The method of claim 8, wherein the surface modification is selected from a lipid, a carbohydrate, a peptide, and combinations thereof.

18. The method of claim 16, wherein the surface modification is PEGylation.

19. A medical article comprising a vessel containing a formulation of MLVs, wherein the formulation contains at least one payload compound, wherein the at least one payload compound is hydrophobic or lipophilic, includes JP4-039, and is ameliorative of at least one of: traumatic brain injury, reactive oxygen species damage, DNA double-strand breaks, and ionizing radiation damage.

20. The medical article of claim 19, wherein the vessel is selected from a vial, a bottle, a syringe, and an autoinjector.

21. The medical article of claim 19, wherein the formulation is a lyophilized medium.

22. The medical article of claim 19, wherein the formulation is a liquid suspension.

23. A method of treating an individual, comprising:
obtaining a medical formulation of MLVs wherein the formulation contains at least one payload compound, wherein the at least one payload compound is hydrophobic or lipophilic, includes JP4-039, and is ameliorative of at least one of: traumatic brain injury, reactive oxygen species damage, DNA double-strand breaks, and ionizing radiation damage; and
administering a dose of the formulation to an individual diagnosed with at least one of: traumatic brain injury, reactive oxygen species damage, DNA double-strand breaks, and ionizing radiation damage.

24. The method of claim 23, wherein the formulation is one of either a lyophilized medium or a liquid suspension.

25. The method of claim 23, wherein the formulation is packaged in a selected from a vial, a bottle, a syringe, and an autoinjector.

26. The method of claim 23, further comprising preparing a dose of the formulation, wherein preparing comprises reconstituting a lyophilized medium of the formulation.
